(19) United States
(12) Patent Application Publication
(10) Pub. No.: US 2016/0317006 A1
(43) Pub. Date: Nov. 3, 2016

(54) SURGICAL ANTIFOGGING DEVICE

(71) Applicant: COVIDIEN LP, Mansfield, MA (US)

(72) Inventors: Ricardo Gomez, Lighthouse Point, FL (US); Sandy Heck, Los Angeles, CA (US); Eric Conley, South Berwick, ME (US); Sean-Michael Jarrett, Royal Palm Beach, FL (US)

(73) Assignee: COVIDIEN LP, Mansfield, MA (US)

(21) Appl. No.: 15/106,038

(22) PCT Filed: Dec. 22, 2014
(86) PCT No.: PCT/US2014/071836

§ 371 Date: Jun. 17, 2016

Related U.S. Application Data
(60) Provisional application No. 61/918,931, filed on Dec. 20, 2013.

(51) Int. Cl. A61B 1/12; A61B 1/313; A61B 1/018; A61B 1/04; A61B 1/00

(52) U.S. Cl. CPC A61B 1/127; A61B 1/04; A61B 1/00163; A61B 1/018; A61B 1/313; A61B 1/00131; A61B 1/00128

(57) ABSTRACT

An antifogging shield prevents fogging and condensation during surgery at a junction of surgical devices. The antifogging shield includes an antifogging shield body forming an interior chamber for accommodating a surgical tool therein and an insulation material disposed within the interior chamber to minimize temperature change.

### CROSS REFERENCE TO RELATED APPLICATIONS

This application is a U.S. National Stage Application filed under 35 U.S.C. §371(a) of International Patent Application Serial No. PCT/US2014/071836, filed Dec. 22, 2014, which claims the benefit of and priority to U.S. Provisional Patent Application Ser. No. 61/918,931 filed Dec. 20, 2013, the content of each of which is incorporated herein by reference.

### TECHNICAL FIELD

The present invention relates to the field of minimally invasive surgery and, more specifically, to fluid management during surgery.

### BACKGROUND

Many surgeries involve the insertion of a surgical tool, for example, a laparoscopic device, into a patient. One of the most common problems affecting surgeons is obstruction of the surgical tools, such as laparoscopic lenses, during surgery. When the lens became obstructed, the surgeon could no longer see and the surgical tool had to be extracted for cleaning. Such extractions of surgical tools delay the overall operating procedure and may cause the patient to receive more anesthesia, expose the patient to increased risk of infection, and lead to higher costs. There is a need for reducing surgical tool obstructions such as the fogging of laparoscopic imaging lens to minimize tool extraction and cleaning during surgery.

### SUMMARY

An antifogging shield may prevent fluids from entering into the camera/scope junction during a surgical procedure. The antifogging shield may prevent fogging from occurring at the camera/scope junction by thermally insulating and liquid proofing the camera/scope junction. Fogging is a major concern in minimally invasive surgeries. Fogging can be caused in several ways. One way is the spilling of liquids right on the laparoscopic lens obscuring the view and requiring the physical cleaning and drying of the lens. A second way that fogging may occur is by rapid temperature changes occurring near the camera/lens junction causing condensation. When a warm fluid touches a colder surface area, rapid temperature changes may occur. When this happens near the laparoscopic lens, fogging quickly follows.

These and other aspects, features and advantages of the present invention will become more readily apparent from the attached drawings and the detailed description of the preferred embodiments, which follow.

### BRIEF DESCRIPTION OF DRAWINGS

Various exemplary embodiments will hereinafter be described in conjunction with the appended drawings, in which:

FIG. 1 is a front view of an antifogging shield;

FIG. 2A is a bottom view of the antifogging shield of FIG. 1;

FIG. 2B is a top view of the antifogging shield of FIG. 1;

FIG. 3 is a cross-sectional view of the antifogging shield of FIG. 2A taken along line 3-3 showing insulating material;

FIG. 4 is a schematic view of the antifogging shield of FIG. 1 and of a laparoscope with a light source;

FIG. 5 is a schematic view of the antifogging shield covering a camera and laparoscope junction of FIG. 4;

FIG. 6 is a schematic view of the antifogging shield covering the camera and laparoscope junction and light source connector cable of FIG. 4 in a different configuration during surgery;

FIG. 7A is a plan view of another embodiment of the antifogging shield in an open position;

FIG. 7B is a bottom view of the antifogging shield of FIG. 7A in a closed position;

FIG. 7C is a top view of the antifogging shield of FIG. 7A in a closed position; and

FIG. 8 is a front view of an alternative embodiment of the antifogging shield.

Like reference numerals refer to like parts throughout the several views of the drawings.

### DETAILIED DESCRIPTION

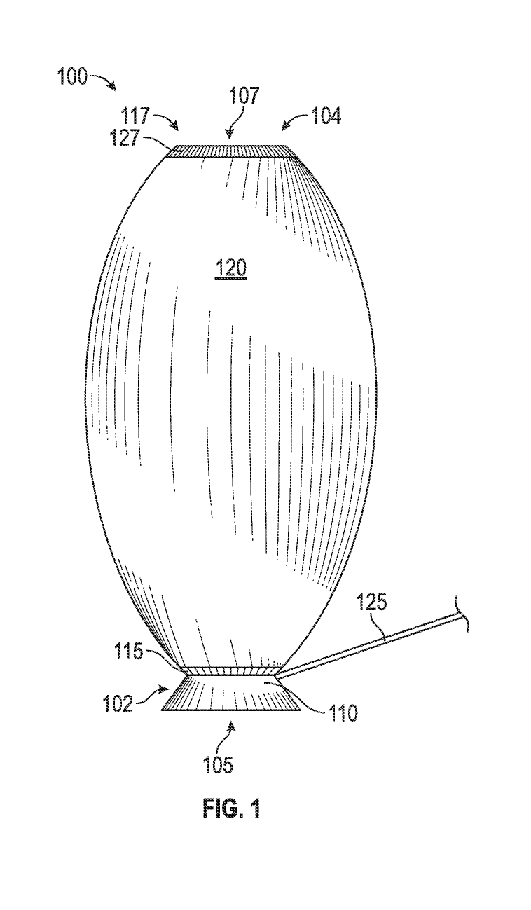

The following detailed description is merely exemplary in nature and is not intended to limit the described embodiments or the application and uses of the described embodiments. As used herein, the word “exemplary” or “illustrative” means “serving as an example, instance, or illustration.” Any implementation described herein as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure, which is defined by the claims. For purposes of description herein, the terms “upper”, “lower”, “left”, “rear”, “right”, “front”, “vertical”, “horizontal”, and derivatives thereof shall relate to the invention as oriented in FIG. 1. Furthermore, there is no intention to be bound by any expressed or implied theory presented in the preceding technical field, background, brief summary or the following detailed description. It is also to be understood that the specific devices and processes illustrated in the attached drawings, and described in the following specification, are simply exemplary embodiments of the inventive concepts defined in the appended claims. Hence, specific dimensions and other physical characteristics relating to the embodiments disclosed herein are not to be considered as limiting, unless the claims expressly state otherwise.

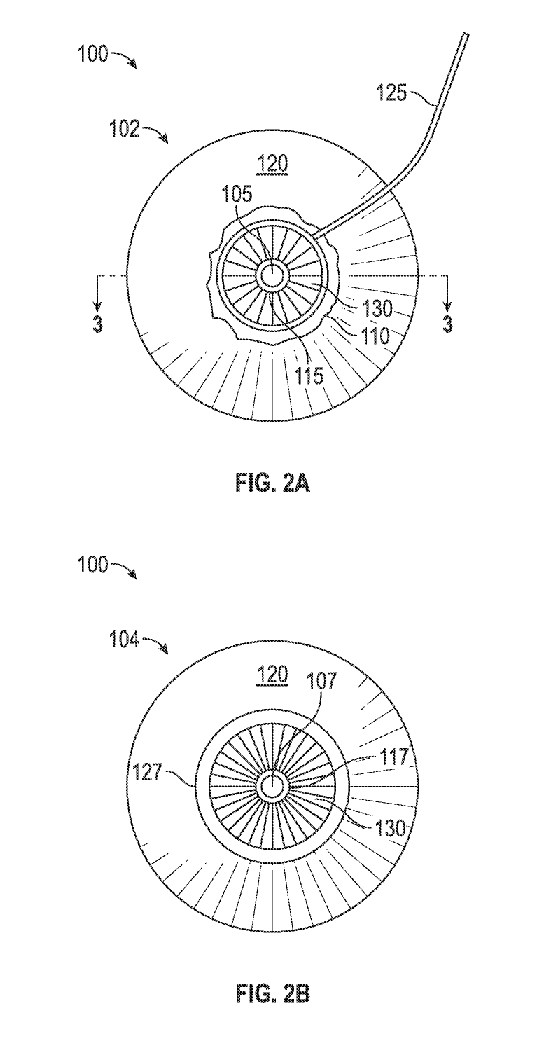

Referring to FIG. 1, an antifogging shield 100 includes a flexible antifogging shield main body 120 extending from a distal end 102 to a proximal end 104. The distal end 102 includes a distal opening 105 formed therein and a first distal elastic band 115 and a self-locking elastic band 125. The proximal end 104 includes a proximal opening 107 formed therein and a first and second proximal elastic bands 117, 127, as shown in FIGS. 2A and 2B. A flexible waterproof insulating guard 130 is disposed around the distal opening 105 and the proximate opening 107. The distal end 102 further includes a flanged end 110 extending outwardly past the distal opening 105 with the self-locking elastic band 125 surrounding the flanged end 110 outward of the distal elastic band 115.

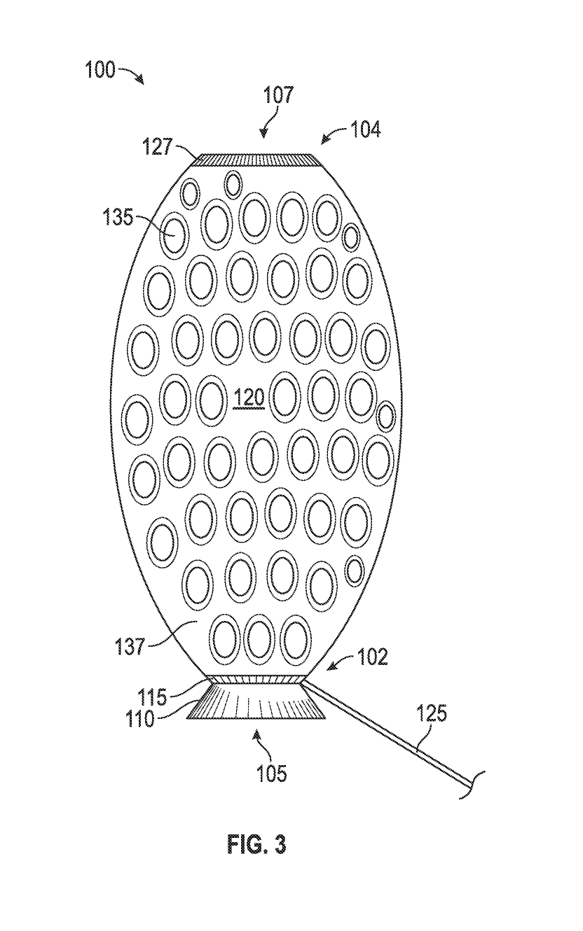

Referring to FIG. 3, the antifogging shield 100 further includes an interior chamber 137 formed within the main body 120 with insulating materia1135 disposed within the interior chamber 137. One type of insulating material can be an air bubble insulation.

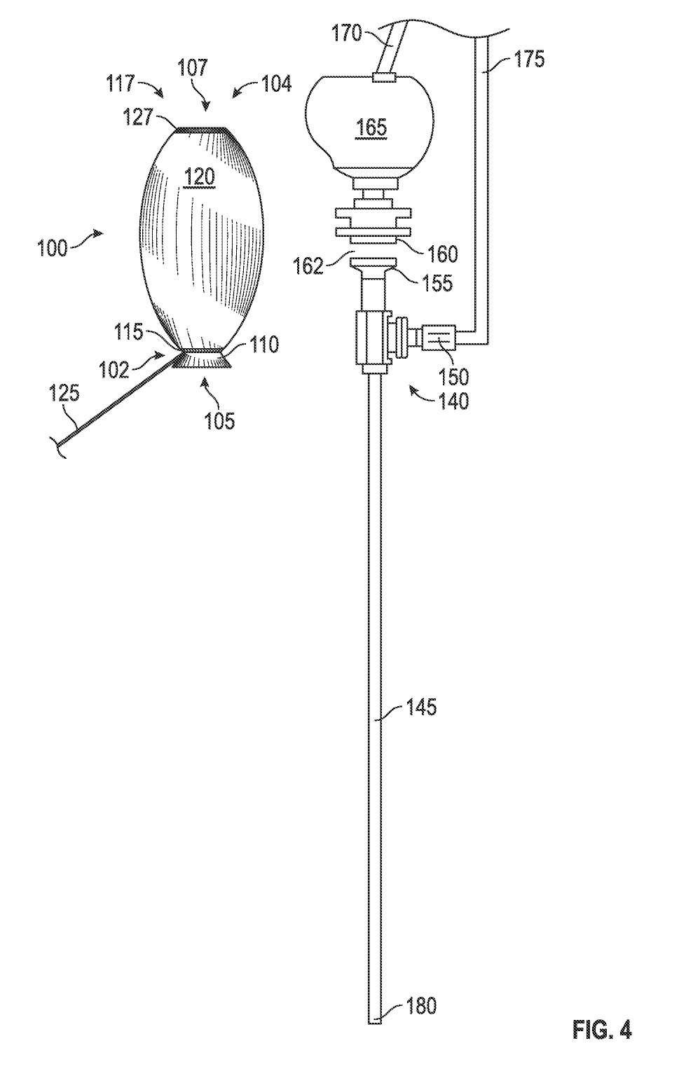

Referring to FIG. 4, the antifogging shield 100 is used during surgeries with a surgical tool 140, such as a laparoscope, for example, and a laparoscopic camera 165. A typical laparoscope usually includes a distal laparoscope end 180 having a laparoscope extension 145 formed therein and a laparoscopic lens 155 disposed on the opposite end of the laparoscope 140. A typical laparoscope may also include a light source connector 150 with a light source cable 175 extending therefrom. The laparoscopic camera 165 includes a camera locking mechanism 160 and a laparoscopic camera cable 170. The camera 165 and the laparoscopic lens 155 define a lens-camera junction 162.

In operation, the antifogging shield 100 forms a liquid proof environment to protect the lens-camera junction 162 of the laparoscopic lens 155 and camera locking mechanism 160. In addition to providing a liquid free environment to the surgical device lens 155, the air bubble insulation 135 helps to prevent rapid temperature changes, which are associated with fogging the laparoscopic lens and lens-camera junction 162. Although air bubbles are shown, any insulating material can also be used to prevent temperature changes.

Elastic bands 115, 125 and 117, 127 restrict the openings 105, 107, respectively, of the antifogging shield 100 to prevent liquid from entering the interior chamber 137. The distal elastic band 115 helps to compress the distal opening 105 and prevent liquids from entering the inner chamber of the antifogging shield 100. Self-locking elastic band 125 is wrapped around flanged end 110 providing an airtight/liquid tight seal. The flanged end 110 extends outwardly from the main antifogging shield 100 providing an extended area where the self-locking elastic band 125 is wrapped around. The second proximal elastic band 127 with a wider opening is used at the proximal end 104. The second proximal elastic band 127 provides a tighter insulated liquid proof fit thus permitting the laparoscopic camera cable 170 and the light source cable 175 to freely pass through.

In operation, the laparoscope 140 is inserted through proximal opening 107, and into the interior chamber 137 with its distal end 180 extending out through the distal opening 105. In the configuration shown in FIG. 4, the antifogging shield 100 will protect the main components from liquids and rapid temperature changes up to the light source connector 150. Proximal elastic band 117 helps to keep the antifogging shield 100 tight around the laparoscopic camera 165. The laparoscopic camera cable 170 is allowed to protrude through the proximal end 104 and back to the monitor (not shown). Once the antifogging shield 100 is placed in the proper position, the self-locking elastic band 125 is wrapped around the flanged end 110 which encompasses the laparoscopic device 140.

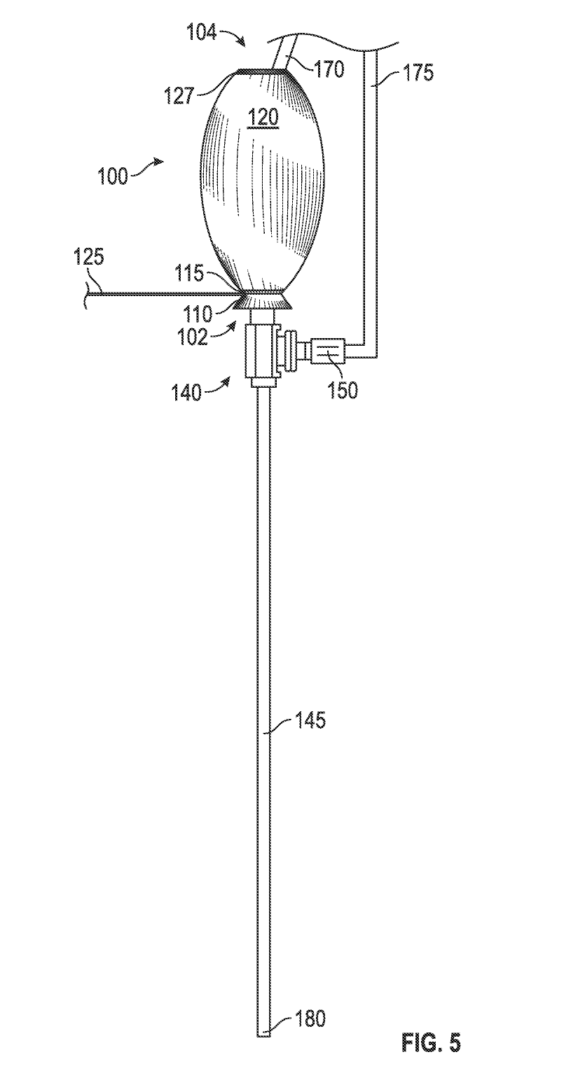

Referring to FIG. 5, the antifogging shield 100 is shown in place protecting the junction of the laparoscopic lens and camera 162. Flanged end 110 provides an additional means of protecting the interior chamber 137 from liquid penetration by providing an area whereby self-locking elastic band 125 is wrapped around it. Self-locking elastic band 125 provides an airtight, liquid tight fit.

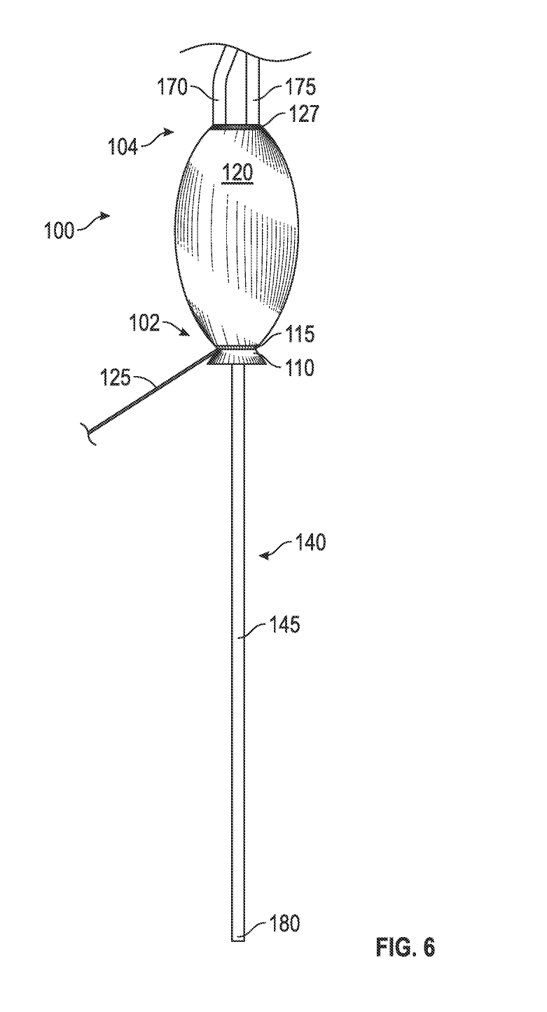

Referring to FIG. 6, a different application of the antifogging shield 100 is depicted whereby the antifogging shield 100 encloses the light source connector cable 150, shown in FIG. 5, and fully protects the main areas of the laparoscopic lens 155 and camera junction. The antifogging shield body 120 acts as a shield against liquid and fogging caused by quick temperature changes attributed to condensation. Once the flexible antifogging shield main body 120 is placed in the correct position, the self-locking elastic band 125 is wrapped around flanged end 110 providing an airtight, liquid tight fit. The laparoscopic camera cable and camera cable 170 extend out through the proximal end 104.

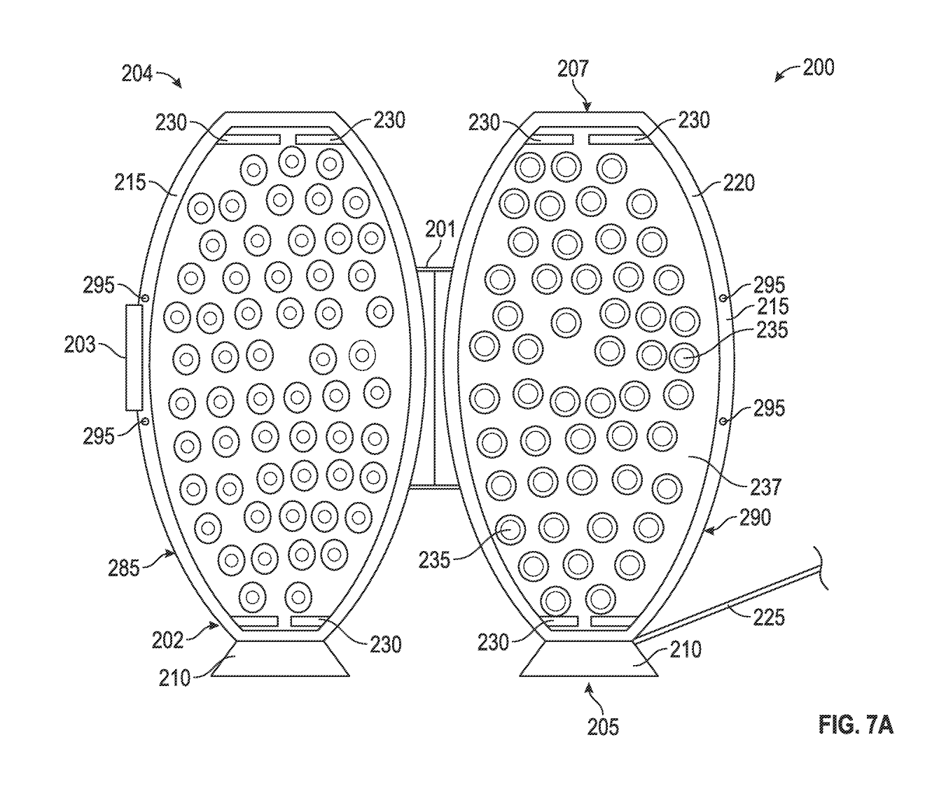

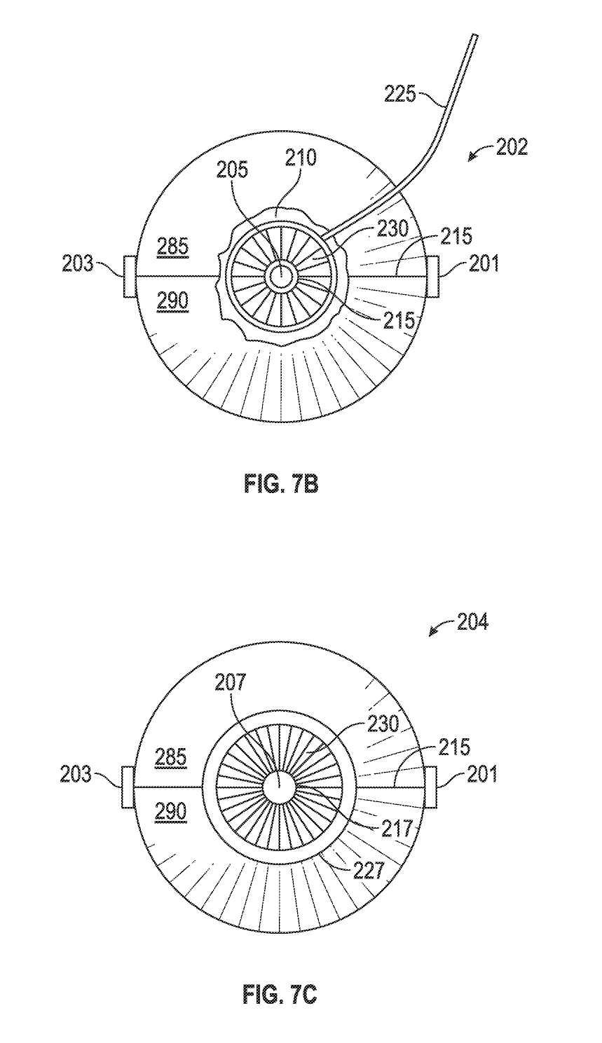

Referring to FIGS. 7A-7C, another embodiment of an antifogging shield 200 includes an antifogging shield main body 220 formed by two hard cover halves 285, 290 connected by a shield connector 201 on one side of the hard cover halves 285, 290. The other sides of the hard cover halves 285, 290 are secured together by a locking mechanism 203 to form the antifogging shield main body 220 and to define an interior chamber 237 therein. The hard cover halves 285, 290 include guide pins 295 disposed at seems 215 to maintain alignment of the hard cover halves. Insulating material 235 is disposed within the interior chamber 237. The insulating material can be any type of insulating material, including air bubbles material.

The antifogging shield main body 220 extends from a distal end 202 and a proximal end 204. The distal end 202, as seen in FIG. 7B, includes a distal opening 205 surrounded by a distal elastic band 215 and flexible waterproof insulating guard 230. The distal end 202 also includes a flanged end 210, with a self-locking elastic band 235 wrapped around the flanged end 210 providing an airtight, liquid tight fit.

As seen in FIG. 7C, the proximal end 204 includes a proximal opening 207 surrounded by a proximal elastic band 217 and flexible waterproof insulating guard 230 and a second elastic band 227 to preclude liquids from entering the inner chamber 237.

In operation, the interior chamber 237 of the antifogging shield 200 accommodates the laparoscope lens and camera junction 162 (shown in FIG. 4) therein. The hard cover halves 285, 290 are closed and locked with the shield connector 201 and locking mechanism 203 to form a single body 220 and a liquid proof environment within the inner chamber 237. Pins 295 guide and ensure that the hard cover halves 285, 290 are properly aligned. Proximal opening 207 and distal opening 205 allow passage of the laparoscope extension 145 to the intended area and permit the laparoscopic camera cable 170 and the light source cable 175 to extend outside of the protected interior chamber 237. The distal elastic band 215 and the proximal elastic band 217 are used at the ends of the hard cover 285, 290 to provide a tight fit to the extending parts. Flanged end 210 helps to provide an additional extension of liquid proofing to the distal end. The air bubble insulation material 235 is used to protect the laparoscopic lens and camera junction 162 from fogging.

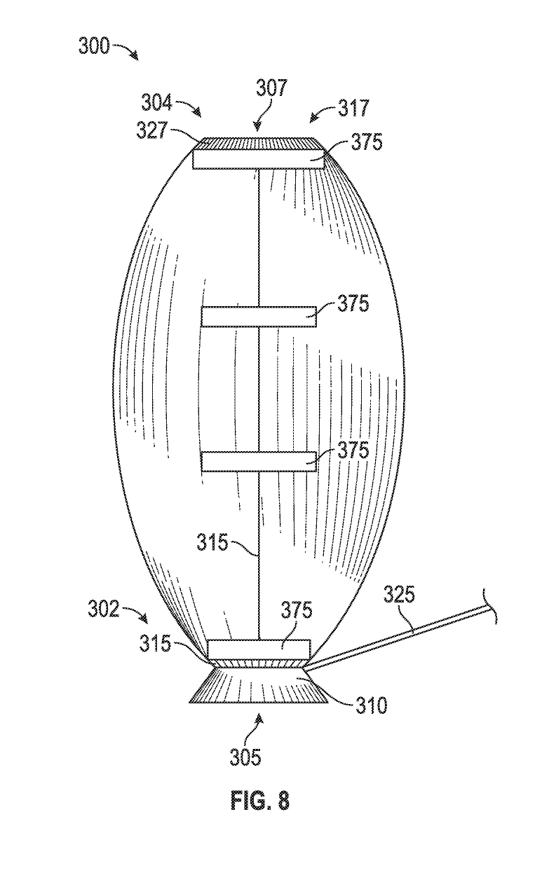

Referring to FIG. 8, an alternative embodiment of an antifogging shield 300 includes hook and loop fasteners 375 to close a seem 315 of flexible main body antifogging shield 320. Several hook and fasteners 375 are used to securely seal the seem 215. The laparoscope 140 and camera locking mechanism 160 are inserted through an opening 307 at a proximate end 304 with their respective camera and light cables 170 and 175 being then inserted through the proximal opening 307. Laparoscope extension 145 is inserted through the distal opening 305 and protected from liquids by the sealing of the flanged end 310, and the distal elastic band 315 and self-locking elastic band 325.

The present invention overcomes the two main problems associated with lens fogging in Arthroscopic and Cystoscopic surgeries.

One advantage of the disclosed device is that the fogging of the lens caused by both liquid spilling into the juncture and condensation caused by the rapid temperature changes caused by the warm liquids coming into contact with the cold medical devices is precluded. The antifogging shield provides both a physical barrier against liquids and a temperature barrier against condensation -the two main causes of lens fogging.

Thus, this device prevents fogging from occurring at the junction of the laparoscopic lens and camera attachment when used in surgical procedures. By preventing fluids from coming into direct contact at the camera-lens junction and thermally insulating them from rapid temperature changes, fogging is prevented from occurring. The antifogging shield addresses both of the previously mentioned problems of fogging caused by liquids spilling into the juncture and by condensation occurring as a result of temperature changes. The disclosed device is a simple and reliable tool used to prevent condensation and fogging at the laparoscopic lens-camera junction.

The antifogging shield disclosed is fabricated from liquid proof material designed to preclude liquids from entering the inner chamber.

As will be recognized by those of ordinary skill in the pertinent art, numerous modifications and substitutions can be made to the above-described embodiments without departing from the scope of the disclosure. Accordingly, the preceding portion of this specification is to be taken in an illustrative, as opposed to in a limiting sense.

What is claimed is:

1. An antifogging shield for use in surgery comprising:
an antifogging shield body forming an interior chamber for accommodating a surgical tool therein; and
an insulation material disposed within the interior chamber to minimize temperature change.

2. The antifogging shield according to claim 1, wherein the antifogging shield body provides a liquid proof environment for preventing fluids from entering the interior chamber.

3. The antifogging shield according to claim 1, wherein the antifogging shield body is flexible.

4. The antifogging shield according to claim 1, wherein the antifogging shield body includes a distal end and a proximal end, wherein each end includes an opening to allow a surgical tool to pass therethrough.

5. The antifogging shield according to claim 4, wherein each opening is sealed to preclude liquid from entering the interior chamber.

6. The antifogging shield according to claim 4, wherein at least one opening is surrounded by an elastic band to preclude liquid from entering the interior chamber.

7. The antifogging shield according to claim 6, wherein the at least one opening comprises a second elastic band to provide additional sealing to the antifogging shield.

8. The antifogging shield according to claim 7, wherein the second elastic band is a self-locking elastic band.

9. The antifogging shield according to claim 1, further comprising a flexible waterproof insulating guard to provide sealing.

10. The antifogging shield according to claim 9, wherein the flexible waterproof insulating guard is disposed about at least one opening formed within antifogging shield body.

11. The antifogging shield according to claim 1, wherein the insulating material is air bubble material.

12. The antifogging shield according to claim 1, wherein the antifogging shield body comprises a hard cover.

13. The antifogging shield according to claim 1, wherein the antifogging shield body comprises two hard cover halves.

14. The antifogging shield according to claim 13, wherein the two hard cover halves are connected to form a waterproof environment within the interior chamber.

15. The antifogging shield according to claim 14, wherein the two hard cover halves are connected by a locking mechanism.

16. The antifogging shield according to claim 14, wherein the two hard cover halves are connected by a hook and loop fasteners.

17. A surgical antifogging device comprising:
an antifogging device body having an interior chamber for accommodating a surgical tool therein, the body providing a liquid proof environment for preventing fluids entering the interior chamber and to minimize temperature change therein.

18. The surgical antifogging device according to claim 17, further comprising an insulation material disposed within the interior chamber.

19. The surgical antifogging device according to claim 17, wherein the antifogging device body is flexible.

20. A surgical antifogging device comprising:
an antifogging device body forming an interior chamber for accommodating a surgical tool therein, the antifogging device body having two openings for allowing the surgical tool to pass therethrough, each of the openings being sealed; and
an insulation material disposed within the interior chamber;
wherein the antifogging device body provides a liquid proof environment for preventing fluids from entering the interior chamber and minimizes temperature change therein.
